(19) United States
(12) Patent Application Publication
(10) Pub. No.: US 2026/0242552 A1
(43) Pub. Date: Aug. 20, 2026

(54) METHOD FOR THE PRODUCTION OF A FILM, A COATING OR A MOULDED BODY FROM YEAST BIOMASS

(71) Applicant: Fraunhofer-Gesellschaft zur Förderung der angewandten Forschung e.V., München (DE)

(72) Inventors: Vanessa WEGAT, Straubing (DE); Michael KOHL, Straubing (DE); Arne ROTH, Straubing (DE); Jonathan FABARIUS, Straubing (DE)

(73) Assignee: Fraunhofer-Gesellschaft zur Förderung der angewandten Forschung e.V., München (DE)

(21) Appl. No.: 19/165,229

(22) PCT Filed: Mar. 14, 2024
(86) PCT No.: PCT/EP2024/056783

§ 371 Date: Sep. 15, 2025

(30) Foreign Application Priority Data
Mar. 17, 2023 (DE) 10 2023 106 741.9

(51) Int. Cl. C08J 5/18; C12N 1/16; C12R 1/645

(52) U.S. Cl. CPC C08J 5/18; C12N 1/16; C08J 2305/00; C08J 2393/00; C12R 2001/645

(57) ABSTRACT

The present invention relates to a method for producing a film, a coating, or a moulded body from yeast biomass, in which the biomass is first disintegrated and homogenized and then further processed into the film, coating, or moulded body. The method is characterized in that an oil-forming yeast, in particular Y. lipolytica, is used as the yeast and the homogenization comprises an enzyme treatment that dissolves the cell wall. The method can be used to produce bio-based and completely biodegradable packaging products that are based solely on renewable and natural raw materials and have good mechanical properties.

### TECHNICAL FIELD

The present invention relates to a method for producing a film, a coating or a moulded body from a biodegradable material, in particular for packaging, in which biomass of a yeast is provided and homogenised by mechanical treatment, and the biomass thus disintegrated is processed to form a film, a coating or a moulded body directly or after addition of one or more further substances.

There is a growing need for biodegradable polymers for the production of product packaging. A sustainable solution is offered by the use of renewable resources, such as plant biomass as a starting material for polymer production. In particular, bio-based polymers have the potential to reduce the use of fossil resources significantly. They are characterized by the fact that they are CO₂-neutral, that is to say they add no or at least very little CO₂ to the atmosphere over their entire life cycle. The bio-based plastics currently established on the market use renewable resources from agriculture. However, this often necessitates using fertile cultivation areas. In contrast, the use of microbial biomass offers the advantage of being a completely renewable source of polymers, fertile agricultural land is not directly needed to provide the biomass and there is no immediate risk of increased deforestation of forest areas for biomass production.

### PRIOR ART

Currently, the main sources of raw materials for producing polymers are renewable resources such as corn, wheat, potatoes, sugar cane, sugar beet, bamboo or wood. Compared with conventional, fossil-based plastics, these bioplastics help to reduce greenhouse gas emissions and can reduce the consumption of fossil raw materials. In some other impact categories (e.g. land and water consumption, eutrophication of bodies of water), however, more often than not net results are worse than for the fossil reference.

Another known possibility is the use of microbial biomass (e.g. yeasts or fungi) for the production of films and coatings in the materials industry. Research and development projects are currently ongoing into the use of the biomass of baker's yeast Saccharomyces cerevisiae in combination with glycerine as a plasticiser, among other things. A corresponding process is described, for example, in J. F. Delgado et al., Characterization of thermal, mechanical and hydration properties of novel films based on Saccharomyces cerevisiae biomass, Innovative Food Science & Emerging Technologies, 2018, 48, pages 240-247. Glycerine improves the integrity, flexibility and mechanical properties of the films produced by acting as a spacer and mechanical mediator between the biopolymer chains at the molecular level. A critical disadvantage of S. cerevisiae, however, is that the wild type is usually cultivated on glucose and as such is dependent on a food-related raw material.

In order to use the yeast components, the cell wall must first be broken down to release the cytoplasm. Delgado et al. use the technique of high-pressure homogenisation at a pressure of more than 100 MPa to produce films from the biomass of S. cerevisiae. The exposed biopolymers can then interact with each other and form a stable network. The next step is thermal treatment in a water bath at 90° C. to denature the proteins and thus increase their interactions with each other. A second homogenisation is used to remove any aggregates (clumps) that may have formed during the heat treatment. Finally, glycerine is added to the yeast dispersion, a film is poured from the mixture and finally the water is removed by drying at 50° C., which further promotes the interaction between the polymer chains and the formation of the film matrix.

Biopolymers from agriculturally produced biomass or the conventionally used microbial sources (e.g. baker's yeast S. cerevisiae) typically interact strongly with water. Consequently, the correspondingly manufactured films have weaker water vapour barriers than synthetic polymers, as a result of which the packaged article dries out faster.

The object of the present invention is to provide a method for the production of a film, a coating or a moulded body that is 100% bio-based and biodegradable. The product produced should be able to be manufactured from renewable and natural raw materials and be suitable for a wide range of applications in the food and packaging industry, and therefore also have sufficient barrier properties for food packaging.

### DESCRIPTION OF THE INVENTION

The object is achieved with the method according to Claim 1. Advantageous variants of the method are the subject matter of the dependent claims or may be discerned from the following description and exemplary embodiment.

The suggested method is based on the use of the biomass of a yeast to produce the film, coating or moulded body, hereinafter also referred to as product or manufactured product. In this context, biomass is understood to mean the complete moist cell mass of the yeast cells, which typically arises in a fermentation method in which this yeast is used. Yeast biomass is an inexpensive and readily available source of biopolymers with promising properties for the development of biodegradable materials. In the suggested method, yeast biomass is first provided and homogenised, with at least one mechanical treatment being carried out for homogenisation. The appropriately homogenised biomass is then processed optionally after further intermediate steps such as heat treatment and another homogenisation process, directly or after the addition of one or more further substances, to form a film, a coating or a moulded body. The suggested method is characterized in that an oil-forming yeast—hereinafter also referred to as oil yeast—is used as the yeast, and the homogenisation comprises a cell wall-dissolving enzyme treatment. This enzyme treatment is preferably a zymolase treatment. The use of other suitable enzymes such as lysozyme or papain is also possible.

Oil-forming yeasts are understood to mean yeasts which can accumulate more than 20% of their dry cell weight as lipids or triacylglycerides. Examples of oil-forming yeasts which can be used in the suggested method are Schwanniomyces occidentalis, Rhodotorula toruloides or Yarrowia lipolytica(Y. lipolytica).

In order to provide the moist biomass of the oily yeast, at least one strain of oily yeast is cultivated. In the suggested method, this cultivation is preferably carried out on crude glycerine as substrate. For example, the glycerine that remains as residue from the production of biodiesel from fats and oils, and which may contain impurities, can be used as a substrate for cultivating the oil yeast used in the suggested method. Instead of crude glycerine, other substrates that are comparable in terms of economic efficiency and resource conservation, such as (used) cooking oils and fats, can be used. This can significantly improve the economic efficiency of the production method, as purification costs for the substrate can be saved. By using side streams such as crude glycerine, these are not only returned to the cycle of materials but also refined (upcycling). The use of food-related substrates such as sugars is not necessary. The production of the product is resource-conserving, the provision of yeast biomass as a raw material does not lead to increased use of fertile and ecologically valuable areas and therefore has no negative impact on biodiversity and is not associated with increased water consumption.

The suggested method can be used to produce films, coatings and moulded bodies that are 100% bio-based and biodegradable. These products have a wide range of applications in the food and packaging industry, are completely compostable and are made solely from renewable natural raw materials. It may also be used to produce packaging products with adequate barrier properties, especially with regard to oxygen and water, as is desired in the food packaging industry. Another advantage of using oil yeasts consists in that, unlike comparable starting materials of this type, improved mechanical properties of the films are achieved. These improved mechanical properties are due to the high lipid and polysaccharide content of the oily yeast. Moreover, the proteins from the yeast cytoplasm and the polymers of the cell wall (polysaccharides such as mannan, glucan and chitin) are particularly relevant for film formation. Compared with the yeast S. cerevisiae mentioned above, an oily yeast has a generally higher polysaccharide content and also a significantly higher chitin content.

The suggested method is implemented particularly effectively with the oily yeast Yarrowia lipolytica (Y. lipolytica). This yeast is able to synthesise a large group of valuable metabolites. Important products are in particular lipases and other hydrolytic enzymes, lipids, citric acid, erythritol and γ-decalactone. Many commercial processes based on the fermentation of the yeast Y. lipolytica also have GRAS (“generally recognized as safe”) status, which is awarded by the Food and Drug Administration. Moreover, the yeast biomass of Y. lipolytica has been approved as a novel food, and it is therefore to be expected that films or similar made therefrom will also be approved for use as packaging material for food without any difficulties. These packaging films are versatile membranes that may only require the use of additives to adjust the integrity and the mechanical properties required for the respective application.

Another important requirement for packaging materials, besides functionality and recyclability, is the protection of the product that is to be packaged. In the food industry in particular, reducing the exchange of water between the food to be protected and its environment is essential. Preventing the packaged articles from drying out is a particularly important objective here, and suitable packaging materials are needed for this. Biopolymers from agriculturally produced raw materials or the microbial biopolymers currently used typically interact strongly with water, and the films made from them form weaker water vapour barriers than in the case of synthetic polymers. A weak water vapour barrier in packaging typically leads to the packaged goods drying out more quickly. Oil-forming yeasts such as Y. lipolytica have a significantly higher lipid content in their cytoplasm (between 20 and 30% of the dry mass, depending on the strain and growth conditions) than other microorganisms. Although lipids alone are unsuitable for forming matrices with acceptable mechanical properties, they are already being used successfully to reduce the strong interaction of films and coatings with water and to improve the water barrier properties of the films. Due to the advantageous composition of biomass from an oily yeast, in particular Y. lipolytica, with its high content of lipids and biopolymers, the requirement for reduced water solubility and consequently also lower water permeability is fulfilled.

In the suggested method, it is particularly advantageous not to use a high-pressure homogeniser for homogenisation and cell disintegration, but rather a mechanical treatment with ultrasound in conjunction with enzyme treatment of the biomass before and/or during the ultrasound treatment. In this regard, it was found that significantly better homogenisation results are obtained with the combination of enzyme treatment and ultrasound treatment than with high-pressure homogenisation. It is also advantageous to subject the biomass to thermal treatment, in a water bath for example, after this first homogenisation step in order to denature proteins in the biomass. This is preferably followed by a further homogenisation step, preferably also using ultrasound treatment.

A further advantageous variant of the suggested method, also in conjunction with the variants described earlier, consists in subjecting the biomass to lyophilisation after cultivation and optionally one or more washing steps. Freeze-drying allows the biomass to be stored for a longer period before further processing according to the suggested method.

Finally, the oily yeast biomass processed according to the suggested method is processed further to form the desired product, i.e. the film, coating or moulded body. For this purpose, one or more further substances may be added to the biomass, which in the method is present in a correspondingly fluid form, in particular as a dispersion in a solvent such as water. This or one of these further substances may be a suitable plasticiser, for example glycerine, to influence the mechanical properties of the manufactured product. It is also possible to add other substances, for example fillers suitable for the respective application. The oily yeast biomass, in particular Y. lipolytica, still constitutes the main component of the manufactured product, for example the film or coating, i. e. a proportion (in percent by weight) of over 50%, typically well over 50%, in particular 70-80% or more. The corresponding product can be manufactured in known manner, for example by simply pouring and drying with regard to production of a film, for example by spraying and drying with regard to production of a coating, or also by injection moulding with regard to production of a moulded body.

### BRIEF DESCRIPTION OF THE DRAWINGS

In the following text, the suggested method will be explained in greater detail with reference to an embodiment in in conjunction with the drawing. In the drawing:

FIG. 1a/b show the growth of two different strains (DSM3286 and PO1F) when cultivated on different glycerine batches;

FIG. 2 shows measurements on films produced with the method with different additions of glycerine; and

FIG. 3 is a diagram of an exemplary process sequence for the production of a film according to the suggested method.

### WAYS TO IMPLEMENT THE INVENTION

The suggested method will explained again in the following text using the preferred oily yeast Y. lipolytica. This oily yeast can use crude glycerine as the sole carbon source for the formation of biomass, resulting in the production of a high proportion of lipids and polysaccharides. In this example, substrates of crude glycerine of different qualities and pure glycerine as a reference were used for cultivation in combination with two different Y. lipolytica strains (DSM3286 and PO1F). The crude glycerine was obtained from various biodiesel manufacturers. Glycerine 1 had a purity of >99%, glycerine 2 a purity of about 90%, glycerine 3 a purity of about 82%, glycerine 4 a purity of about 70%, and glycerine 5 a purity of only about 50%.

First, the two Y. lipolytica strains were cultivated in a mineral salt medium with the above-mentioned glycerine batches (using 20 g/L starting concentration in each case) as substrates. The Y. lipolytica strain PO1F is a genetically modified version of the wild-type strain W29, which is used frequently in the laboratory or in industrial processes. In this case, genes for leucine and uracil synthesis were deleted in order to establish auxotrophies as selection markers for genetic engineering processes. Strain DSM3286 is a wild-type strain which comes from the culture collection of the DSMZ. The crude glycerines were not specially prepared for cultivation, but simply sterilely autoclaved and then used directly. Cultivation was carried out at a temperature of 30° C. at 200 rpm for 48 hours. The biomass was then harvested by centrifugation (8,000 g, 10 min, 4° C.).

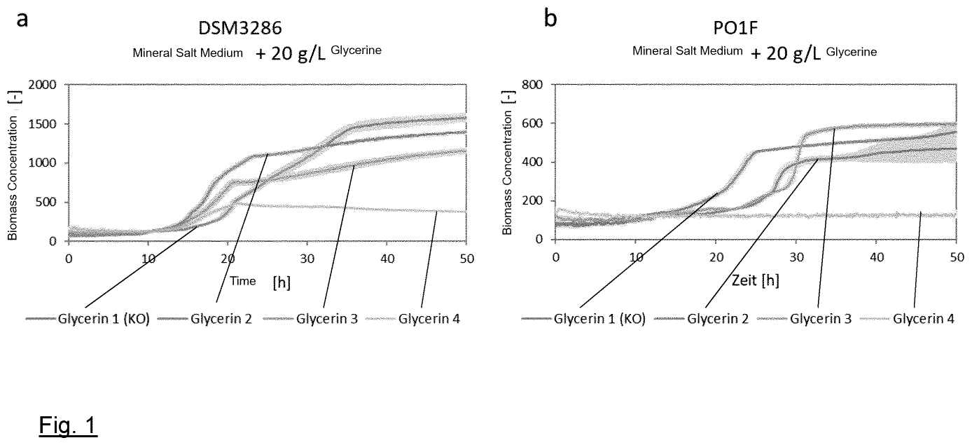

It was found that both strains tested can make use of all the glycerine sources used, wherein strain PO1F achieved lower biomass yields than strain DSM3286. Depending on the strain and glycerine batch, the maximum biomass concentration could be reached under the conditions tested here in a timeframe between 24 hours and 36 hours. Biomass formation over time with the two strains in the mineral salt medium and the respective glycerine batches was recorded over 48 hours in a parallel microbioreactor. FIG. 1 shows the measured biomass concentrations as a function of time for the substrates glycerine 1 to glycerine 4 and the two strains used, DSM3286 (FIG. 1a) and PO1F (FIG. 1b). The use of glycerine 5 results in significant foaming, which meant that a reliable measurement of the optical density to determine biomass concentration was not possible. However, both strains were able to use Glycerine 5 as a growth source despite its low purity.

As may be seen from FIG. 1, the strain DSM3286 shows a significantly higher biomass yield than the strain PO1F and is therefore more suitable for industrial use. In addition, DSM3286 is not a genetically modified organism (GMO) and achieves a high lipid yield naturally. The following experiments were therefore carried out with biomass obtained from this strain, using glycerine 2 and glycerine 3 as the crude glycerine source for cultivation.

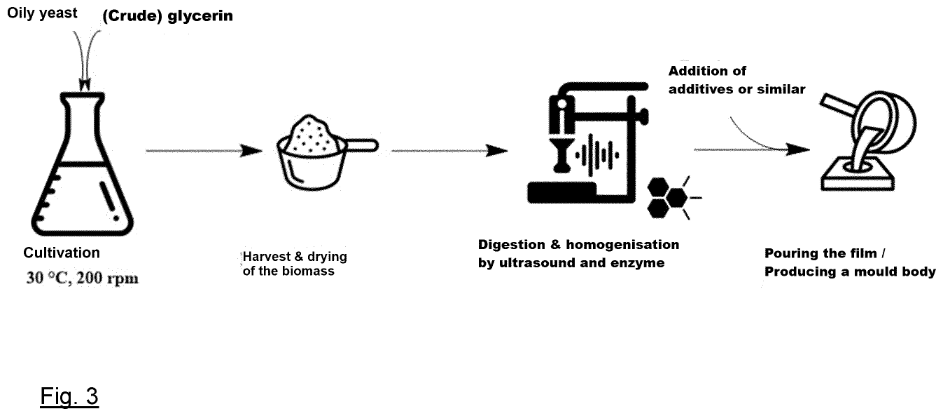

When conducting experiments to produce films from this biomass, it was found that the homogeneity of the film can be improved if the following biomass digestion method is used instead of high-pressure homogenisation. Instead of high-pressure homogenisation, the biomass was homogenised in an ultrasonic sonotrode for 5 minutes each, after being treated with Zymolase for one hour at 37° C. Zymolase contains a group of enzymes which, among other things, hydrolyse the β-1, 3-glucane bonds contained in the cell wall, thus ensuring improved decomposition and consequently digestion of the yeast cell wall. This procedure made it possible to produce more uniform films, no longer containing intact yeast cells, than with the high-pressure homogenisation that has generally been used up to now. The use of ultrasound for homogenisation is not absolutely necessary to solve the present problem. Other mechanical homogenisation processes can also be used for this purpose. However, the use of ultrasound in conjunction with the Zymolase treatment offers the additional advantage described above of producing more uniform films. The entire method from cultivation to the production of films or a moulded body is represented for exemplary purposes in the diagram in FIG. 3.

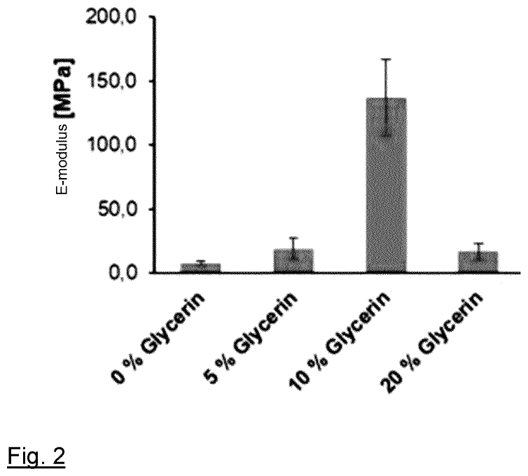

The films were then examined to determine the film properties, for example by means of differential calorimetry and determination of the E-modulus. The elastic modulus (E-modulus) is a parameter for how much a material gives way when force is applied (resistance to deformation). For the same load and geometry, a component made from a material with a high elastic modulus (such as steel) is more rigid than the same component made from a material with a low elastic modulus (such as rubber). For this purpose, tests were carried out with different glycerine contents in the films. Of the glycerine contents tested (0%, 5%, 10% and 20%), the best E-modulus was achieved with 10% glycerine, as can be seen from FIG. 2. Differential calorimetry analyses showed a melting point of about 90° C.

Furthermore, pressed pieces were also produced from layered foils. This shows that the foils can be processed into a firmly bonded workpiece and are therefore suitable for the pressing process. For this purpose, several plies of the films were cut to size and layered according to the pressing mould. The pressed pieces were moulded at a temperature of 120° C. and a time of 80 seconds with final cooling.

The products manufactured using the suggested method are also readily compostable. This was proven in tests. Depending on the conditions and the thickness of the material, complete composting takes about 4 to 8 weeks. The key to the product's excellent compostability lies in its natural ingredients. Unlike many bioplastics, the films manufactured using the method are based on natural biopolymers (i.e. yeast biomass and glycerine). Thus, the microorganisms in nature already have the enzymes they need to break down these compounds and use them as substrates for their natural metabolism. This means that biomass is once again built up in the environment and the carbon cycle is closed.

The barrier properties of the films produced using the method are also sufficient for applications in the food sector. For example, measurements on films produced using the present method with 10% glycerine showed a standardised oxygen permeability measured at 23° C. /50% RH in the range from 36 to 41 cm³ 100 μm/(m² d bar) and an average standardised water vapour permeability measured at 23° C./85% RH of 1409±227 g 100 μm/(m² d).

Natural polymers such as proteins and polysaccharides tend to have a rather low barrier due to their high number of hydrogen bonds to water vapour but show a high barrier effect against oxygen. With lipids it is usually the other way around: the hydrophobicity results in a good barrier in respect of water vapour but shows high permeability to oxygen. In the film produced according to the suggested method, the polysaccharide and lipid contents can be additionally controlled during fermentation, e.g. by media composition, fermentation conditions (duration, temperature, agitation rate, etc.) or by genetic modifications to the yeast. The properties of the products produced with the method may be influenced further by modifying the yeast biomass, to improve packaging-relevant functionalities, for example. The production rate and yield of yeast biomass depend heavily on the concentration of the carbon and nitrogen sources as well as on the aeration of the cultivation medium and the cultivation temperature. Above all, the composition of the medium significantly influences the composition of the biomass and in particular its lipid content. Thus, the properties of the products produced can be further optimised by influencing these parameters, in particular by the composition of the medium and the fermentation conditions.

1. Method for producing a film, a coating or a moulded body from a biodegradable material, in particular for packaging, which comprises at least the following steps:
Providing biomass of a yeast,
Homogenising the biomass, which comprises at least one mechanical treatment of the biomass, and
Processing the homogenised biomass directly or after addition of one or more further substances to form a film, a coating or a moulded body,
characterized in that
an oil-forming yeast is used as yeast, and the homogenisation also comprises a cell wall-dissolving enzyme treatment.

2. Method according to claim 1, characterized in that the provision of the oil-forming yeast comprises a cultivation of one or more strains of said yeast on crude glycerine as a substrate.

3. Method according to claim 1, characterized in that Yarrowia lipolytica is used as the oil-forming yeast.

4. Method according to claim 1, characterized in that the enzyme treatment is carried out by a zymolase treatment.

5. Method according to claim 1, characterized in that the mechanical treatment is carried out by means of ultrasound.

6. Method according to claim 1, characterized in that glycerine is added as the further substance or one of the further substances.

7. Method according to claim 1, characterized in that after cell disintegration, the biomass undergoes a thermal treatment, preferably in a water bath, in order to denature proteins in the biomass.

8. Method according to claim 7, characterized in that following the thermal treatment, a further homogenisation of the biomass is performed.

9. Method according to claim 1, characterized in that the biomass is freeze-dried before homogenisation.

10. Use of the biomass of an oil-forming yeast, in particular of Yarrowia lipolytica, for the production of a film, a coating or a moulded body.

11. Film, coating or moulded body which are formed as the main component from the biomass of an oil-forming yeast, in particular Yarrowia lipolytica.
